(19) United States
(12) Patent Application Publication
(10) Pub. No.: US 2009/0131844 A1
(43) Pub. Date: May 21, 2009

(54) Tension Strap for Migration Prevention and Patellar Stabilization for Use With Knee Braces

(76) Inventors: John Harvey Dean, Birmingham, AL (US); Michael Alan Spooner, Birmingham, AL (US)

Correspondence Address: BRADLEY ARANT BOULT CUMMINGS LLP;INTELLECTUAL PROPERTY DEPARTMENT, 1819 FIFTH AVENUE NORTH, BIRMINGHAM, AL, 35203-2104, US

(21) Appl. No.: 11/943,997

(22) Filed: Nov. 21, 2007

(51) Int. Cl. A61F 5/00

(52) U.S. Cl. 602/36; 602/26

(57) ABSTRACT

A tension strap to be used with any brand of ACL or OA functional knee bracing. The position, elasticity and versatility of the strap allow it to be used for patella support and to prevent vertical migration of the brace. The strap is placed over the anterior side of the face of the knee joint with the support buttress lying adjacent to the anterior edge of the patella and the support straps extended posteriorly to the upper arms and lower arms of the brace. The support straps are then pulled around the outside of the upper arms and lower arms of the brace and adhered back to themselves using hook and loop fastener material. Additional support tension can be applied to the patella through the buttress support by increasing tension through the support straps. The prevention of vertical migration of the knee brace is also achieved through the use of non-slip lining on the face of the tension strap that is in contact with the skin covering the knee joint and patella.

### BACKGROUND OF INVENTION

The present disclosure provides a tension strap for use with knee braces. The knee joint is one of the human body's most vulnerable joints to injury in sport, recreation, and physical labor. The patella femoral joint is a common site of pain and injury. The etiology of patella femoral pain can be traumatic or insidious in nature, but it is commonly the result of lateral force vectors acting on the patella. This lateral force vector can be a result of medial ligamentous injury (documented in studies and articles published by Dr. William P. Garth), tight lateral tissue, or poor biomechanical alignment (increased quadriceps angle, improper landing strategy, etc.).

Several patella femoral braces are available on the market which focus on attempting to control the lateral displacement of the patella. These braces are specifically designed to control the tracking in a single plane for the Medial Patella Meniscus Ligament (MPML). However, the ability to control the MPML and also the Medial Patella Femoral Ligament (MPFL) is not currently available in conjunction with multiple instability rigid bracing. The present invention addresses both these ligament issues.

Osteoarthritis (OA), Anterior Cruciate Ligament (ACL) and other functional braces are widely used by athletes and other individuals to protect the knee from injury, decrease stresses to healing tissue (postoperatively), and to decrease exacerbating factors with existing injuries. Several manufacturers produce functional braces designed to protect ligament tissue of the knee (primarily the ACL, but also the Medial Collateral Ligament (MCL) and the Posterior Cruciate Ligament (PCL)) as well as the articular cartilage of the knee (OA unloading braces). These braces are widely used in sports for the reasons mentioned above as well as for prophylaxis in high velocity/high impact sports (such as, but not limited to, football) and everyday patients with OA knee pain. Common mechanisms of injury these braces protect against include excessive valgus forces (a blow to the lateral knee), rotational forces (commonly internal rotation of the femur on the fixed tibia), shearing forces (anterior shear of the tibia on the femur) and unloading joint compartmental issues. However, these braces are only effective if the brace is properly aligned on the wearer's leg and with the patella femoral joint.

Athletic trainers responsible for keeping these types of braces on the players during sporting events express frustration about the constant adjusting of the functional braces on the athletes during use, and often trainers and athletes resort to such ad hoc measures as taping the brace to the wearer's leg to keep the brace from falling down the leg. It is one object of the present invention to assure that such rigid OA and ACL braces will be in proper alignment while in use. Athletic trainers and coaches who have used prototypes of the present device of the disclosure are enthusiastic about the potential to solve this frustrating migration problem.

A related problem results when applying rigid braces to larger patients. The leg shape often encountered in such patients is referred to as “upside down cone shaped leg” and results in rigid braces of common design sliding down the leg during use. This brace migration can be the result of hinge design not matching physiologic axis of rotation at the knee; muscle atrophy after injury, surgery or immobilization; or poor purchase (or fit) of off-the-shelf functional braces.

The brace migration introduced by inherent design problems in the typical rigid brace is exacerbated by the fact that the rigid brace is supported by contracting, constantly moving muscle tissue. The present device of the disclosure solves the problem of brace migration by capturing the patella and skin of the knee and utilizing the forces that are present throughout flexion and extension keep the brace system suspended in the desired, proper alignment with the knee. The brace migration issues associated with hinge design, muscle atrophy, “upside down cone shape leg” and constantly moving muscle tissue have been overcome in several trials of prototypes of the present invention.

Furthermore, clinical cases often require bracing to address other instabilities of the knee, such as, but not limited to, both patella femoral bracing and OA/ACL bracing. This can occur with an ACL tear or other injury concurrent with patellar subluxation/dislocation where the mechanism of injury is similar (valgus/external rotation of the leg). Another common occurrence is where the patient is performing rehabilitation activities after knee surgery, such as ACL reconstruction, and develops patella femoral pain, which is significant because patella femoral pain can inhibit muscle contraction and result in a difficult, lengthy rehabilitation process. In such instances of patella subluxation occurring in conjunction with multiple instabilities of the knee, it is an object of the present disclosure to provide a tension strap for use on rigid braces existing on the market today to simultaneously control patella instability problems and brace migration issues. Thus, the present disclosure provides a simple, cost effective mechanism to solve the problems of functional brace migration and the lack of existing products in the art that simultaneously address the multiple issues patella and knee instability while also addressing functional brace migration. Significant economies to the user are achieved because the device of the present disclosure can be applied to most any functional brace on the market without permanent modification to the functional brace, a fact most useful in situations where only temporary patella femoral support or anti-migration is needed, such as postoperative rehabilitation, sporting events, larger leg sizes, etc.

### SUMMARY OF THE INVENTION

The present disclosure provides a tension strap for use with knee braces, such as but not limited to, knee braces used for the pre- and post-injury support of the anterior cruciate ligament (ACL). The disclosed tension strap provides for at least one of the following functions: (i) prevention of vertical migration of the knee brace during use; and (ii) providing support to the patella (knee cap) which is not normally provided in existing braces. The disclosed tension strap provides for the prevention of vertical migration by pulling the hinges of the knee brace in towards the knee and through the grip provided by contact between the material of the tension strap and the skin of the user. In addition, the tension strap provides stabilization of and support to the patella by applying corrective forces to the patella to ensure proper patella alignment and dynamic tracking of the patella femoral joint. In one embodiment, the disclosed tension strap performs both functions. However, in an alternate embodiment, the tension strap may provide only one function, such as, but not limited to, prevention of vertical migration of the knee brace.

The tension strap of the present disclosure also provides for increased efficiency in use of the knee brace compared to the methods known in the art. By providing a mechanism to prevent vertical migration of the knee brace, the tension strap eliminates interruptions in physical activity, most notably athletic contests, practices for those contests and rehabilitation activities, where a migrating knee brace has to be refitted to proper placement on the wearer's knee.

### BRIEF DESCRIPTION OF THE DRAWINGS

FIGS. 1a and 1b are, respectively, anterior and posterior views of one embodiment of the tension strap of the present disclosure with buttress support member attached in final position on the knee brace and the knee brace worn on the user's right knee. While the design of the knee brace worn on the right knee is different from the design of the knee brace worn on the left knee, the tension strap is universal in fit for both right knee and left knee braces.

FIGS. 2a and 2b are views, respectively of the bottom and top of one embodiment of the tension strap of the present disclosure with buttress support member.

FIGS. 3a and 3b are, respectively, anterior and posterior views of one embodiment of the tension strap of the present disclosure with buttress support member prior to final attachment of the tension strap onto the knee brace (again illustrating a knee brace worn on the right knee of the user).

FIGS. 4a and 4b are medial side views of one embodiment of the tension strap of the present disclosure showing, respectively, the unlooped and looped medial side support straps (again illustrating a knee brace worn on the right knee of the user).

FIGS. 5a and 5b are lateral side views of one embodiment of the tension strap of the present disclosure with buttress support member showing, respectively, the unlooped and looped lateral side support straps (again illustrating a knee brace worn on the right knee of the user).

FIGS. 6a and 6b are, respectively, anterior and posterior views of one embodiment of the tension strap of the present disclosure without buttress support member attached in final position on the knee brace (again illustrating a knee brace worn on the right knee of the user).

FIGS. 7a and 7b are bottom and top views, respectively, of one embodiment of the tension strap of the present disclosure without a buttress support member.

FIGS. 8a and 8b are, respectively, anterior and posterior views of one embodiment of the tension strap of the present disclosure without buttress support member prior to final attachment of the tension strap onto the knee brace (again illustrating a knee brace worn on the right knee of the user).

FIGS. 9a and 9b are lateral side perspective views of one embodiment of the tension strap of the present disclosure without buttress support member showing, respectively, the unlooped and looped lateral side support straps (again illustrating a knee brace worn on the right knee of the user).

### DETAILED DESCRIPTION OF PREFERRED EMBODIMENTS

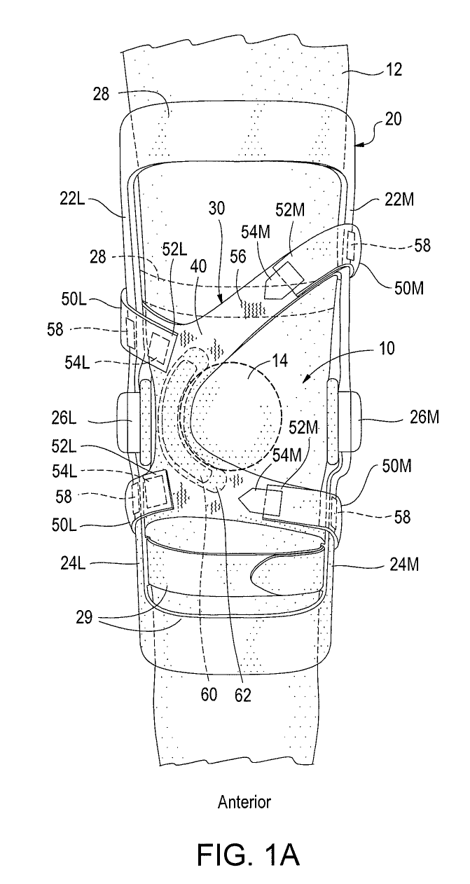

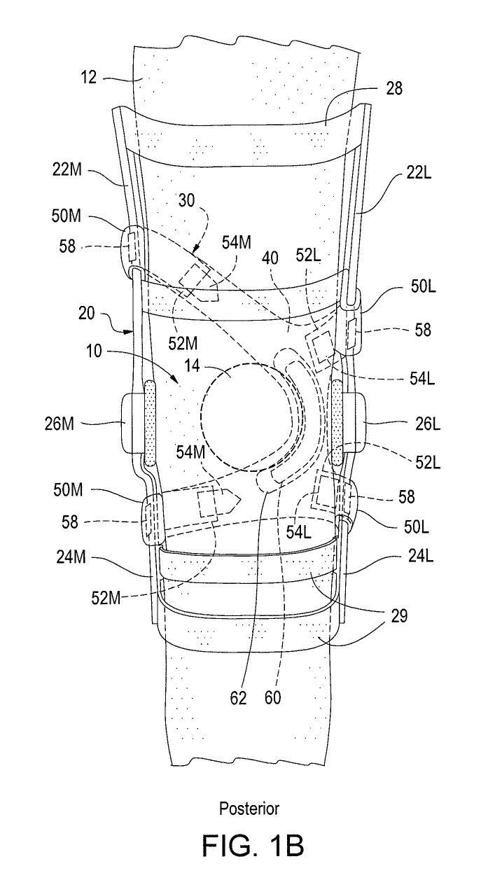

Referring more particularly to the drawings, FIGS. 1 (a & b) and 6 (a & b) show the tension strap 30 used in combination with a knee brace 20 worn on the leg 12 of a user. The knee brace 20 typically includes one or more proximal cuffs 28 engageable with the wearer's leg 12 above the knee 10 and one or more distal cuffs 29 engageable with the wearer's leg 12 below the knee 10. The knee brace 20 typically has a pair of hinge assemblies (26L and 26M) disposed medially (M) and laterally (L) about the joint of the knee 10. The proximal cuffs 28 of the knee brace 20 are typically connected to the pair of hinge assemblies (26L and 26M) by a pair of upper arms (22L and 22M) generally disposed medially and laterally about the knee 10 and leg 12. The distal cuffs 29 of the knee brace 20 are also typically connected to the pair of hinge assemblies (26L and 26M) by a pair of lower arms (24L and 24M) also generally disposed medially and laterally about the knee 10 and leg 12.

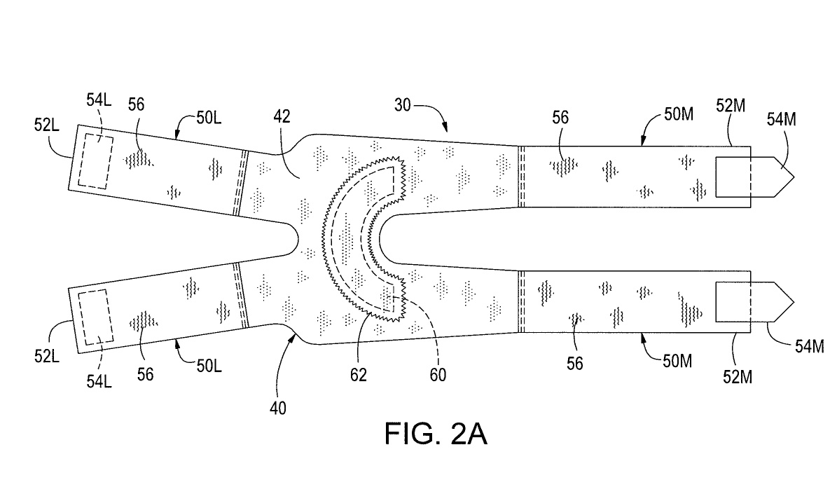

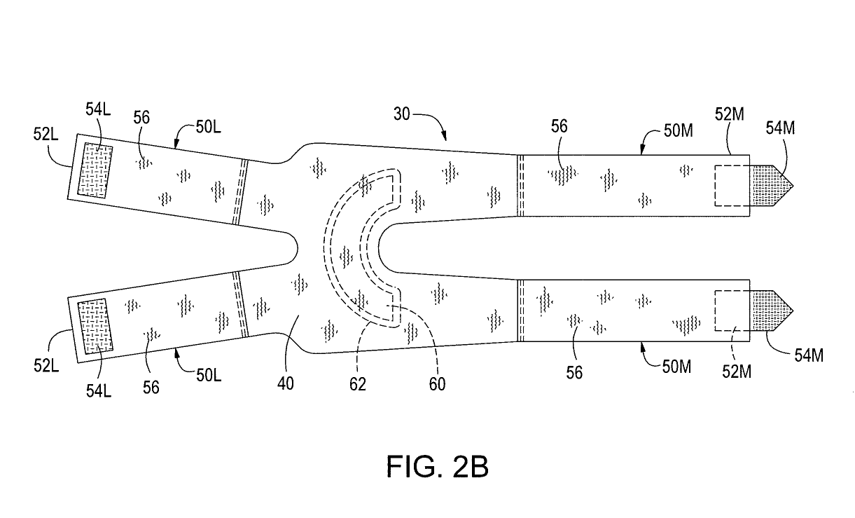

As will be seen in FIGS. 2 (a & b), in one embodiment of the present invention, the tension strap 30 has three primary parts: (i) the body 40, all or part of which engages all or part of the skin on the anterior side of the wearer's knee 10 and patella 14; (ii) a plurality of support straps (50L and 50M) (here shown as four in number) extending distally from the body 40 which engage the knee brace 20 at the distal ends 52L and 52M of the support straps (50L and 50M) in the manner later described; and (iii) a support buttress member 60 for the patella 14.

The body 40 is formed of a flexible, elastic material, such as, but not limited to, neoprene with unbreakable looped backing (ULP) on the outer surface of the body 40. The inner surface 42 of the body 40, being that surface of the body 40 that will contact the skin on the anterior side of the user's knee 10 in the manner later described, is of a non-slip material, such as, but not limited to, exposed neoprene or synthetic shark skin.

The support straps (50L and 50M) are formed of a flexible, elastic material, such as, but not limited to, neoprene with unbreakable looped backing (ULP) on both sides of the support straps (50L and 50M). In one embodiment, the support straps (50L and 50M) and body 40 are affixed together by sewing, however, one skilled in the art will understand that the support straps (50L and 50M) and body 40 may be of unitary construction or may be affixed together by other like means.

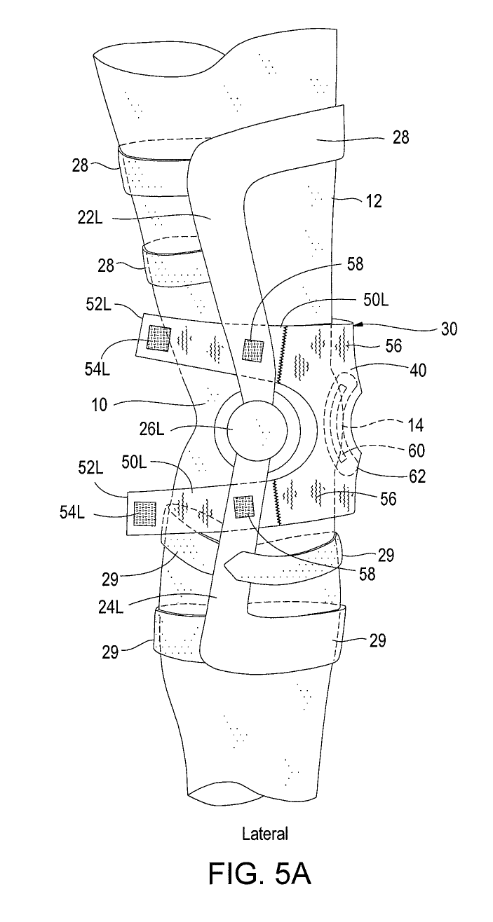

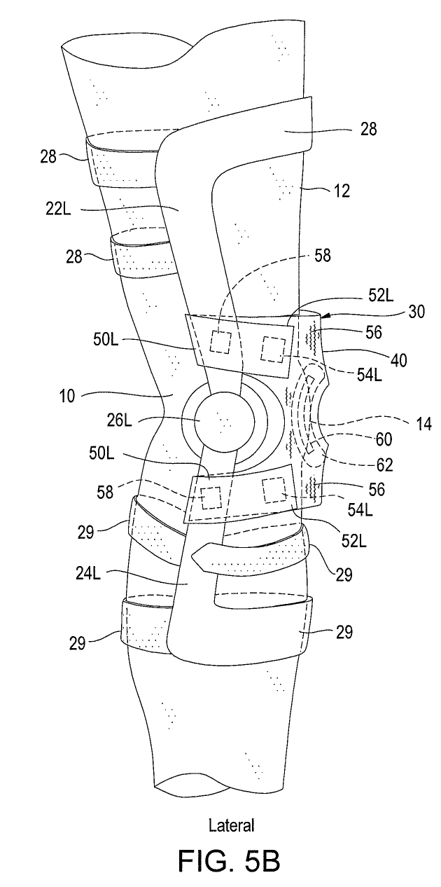

As shown in FIGS. 1a, 2a, 3a and 5a, the buttress support member 60 has a low elevational profile with an arcuate medial profile to substantially conform to the dimensions of the lateral circumference of the patella 14. One skilled in the art will understand that the buttress support member 60 may also take the shape of a complete loop, circular or otherwise, such that the inside dimension of the buttress support member 60 will substantially conform to the dimensions of the entire circumference of the patella 14 and that the dimension of the body 40 and support straps 50 can be modified to accommodate a buttress support member 60 of different shapes. The buttress support member 60 is fashioned from a firm, flexible material, such as, but not limited to, thick rubber tubing. The buttress support member 60 is affixed to the inner surface 42 of the body 40 such that it will contact and provide support to the patella 14 in the manner later described. In one embodiment, the buttress support member 60 is encased within a guide pocket 62 that is arcuately configured with the outer dimensions of the buttress support member 60 and is secured to the inner surface 42 of the body 40 as described above). The exterior surface of the guide pocket 62, being that surface of the guide pocket 62 that will contact the skin on the anterior side of the wearer's knee 10 in the manner later described, may be of the same non-slip material as is found covering the inner surface 42 of the body 40.

As shown in FIGS. 1 (a & b), 2 (a & b), 3 (a & b), 4 (a & b) and 5 (a & b), the lengths of the support straps (50L and 50M) are such to permit the distal ends (52L and 52M) of the support straps (50L and 50M) to be wrapped or looped about the medially and laterally disposed upper arms (22L and 22M) and lower arms (24L and 24M) of the knee brace 20 in the fashion later described. In one embodiment, the support straps 50L to be affixed to the laterally disposed upper arm 22L and lower arm 24L of the knee brace 20 are shorter in distal length than those support straps 50M that will be affixed to the medially disposed upper arm 22M and lower arm 24M of the knee brace 20 such that less distal elastic deformation will be experienced across the lengths of the laterally disposed support straps 50L than that experienced by the medially disposed support straps 50M, thereby anchoring the buttress support member 60 along the lateral aspect of the patella 14 and knee 10 during flexion of the knee 10 in the manner later described.

As shown in FIGS. 2 (a & b), a patch of hook fastener material (54L and 54M) is affixed to the distal end (52L and 52M of each support strap (50L and 50M). In one embodiment, the outer dimensions of the patches of hook fastener material 54L affixed to the distal ends 52L of the support straps 50L are contained within the outer dimensions of the outer surface of the support straps 50L and may be affixed to the outer surface of the support straps 50L by such means as gluing, sewing or through the use of double sided hook fastener material. In one embodiment, the patches of hook fastener material 54M affixed to the distal ends 52M of the medially oriented support straps 50M append distally off the distal ends 52M and are affixed to the support straps 50M by use of alligator tabs also of hook fastener material that adhere to both the inner and outer surface of the support straps 50M. However, it will be appreciated that the patches of hook fastener material 54M may be affixed to the distal ends 52M of the support straps 50M by any manner of acceptable means.

Because loop fastener material 56 is distributed about the proximal surfaces of each support strap (50L and 50M) and the outer surface of the body 40, the support straps 50 (L and M) having been wrapped or looped about the medial and lateral upper arms (22L and 22M) and lower arms (24L and 24M) of the knee brace 20 in the fashion later described, upon closing the looped support straps (50L and 50M), the patch of hook fastener material (54L and 54M) of each support strap (50L and 50M) will adhere to the loop fastener material 56 on the top surface of each support strap (50L and 50M) or the outer surface of the body 40, thereby securing the distal ends (52L and 52M) of the support straps (50L and 50M) about the upper arms (22L and 22M) and lower arms (24L and 24M) of the knee brace 20.

As shown in FIGS. 1(a & b), 3(a & b), 4(a & b) and 5(a & b), a self adhesive patch of hook fastener material (58) will also be applied to each of the upper arms (22L and 22M) and lower arms (24L and 24M) of the knee brace 20 in a desired positions along the upper arms (22L and 22M) and lower arms (24L and 24M) of the knee brace 20 where the support straps (50L and 50M) will be wrapped or looped about and secured to the upper arms (22L and 22M) and lower arms (24L and 24M) of the knee brace 20 in the manner later described so as to anchor the vertical positions of the support straps (50L and 50M) about the upper arms (22L and 22M) and lower arms (24L and 24M) of the knee brace 20.

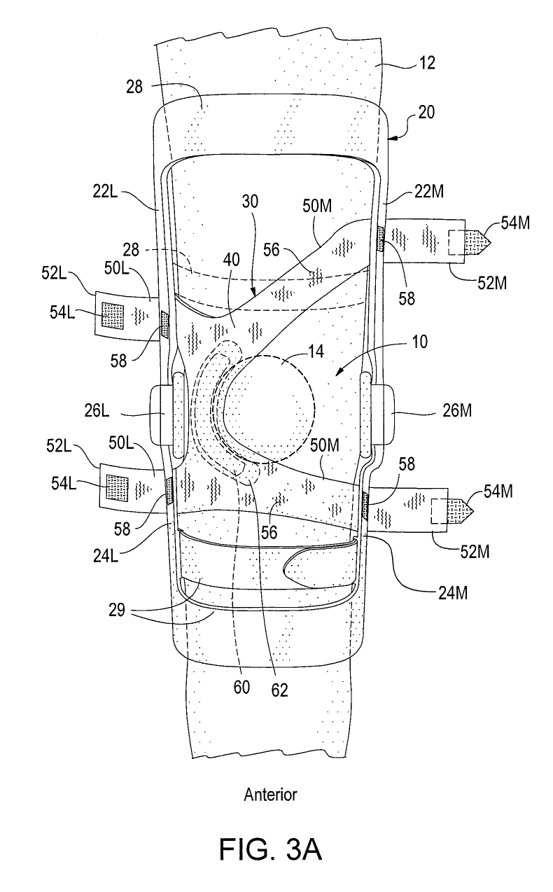

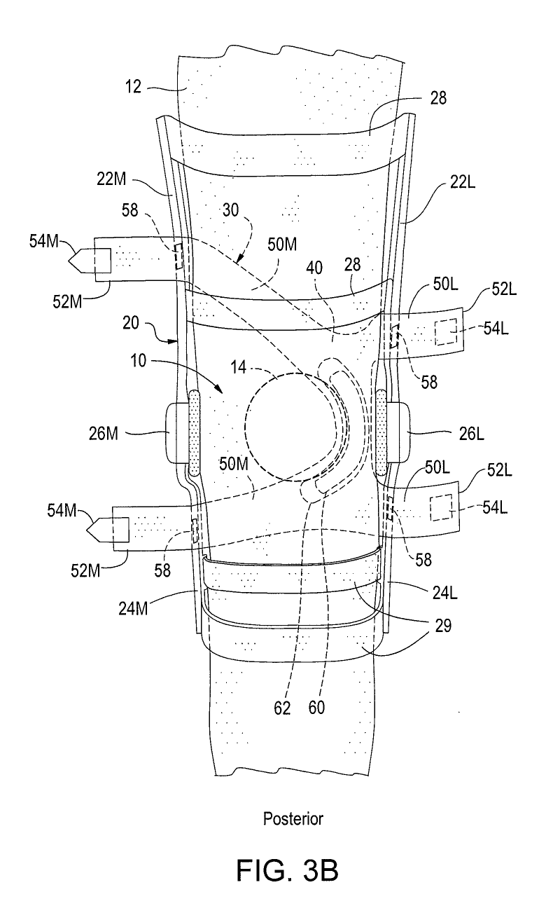

Operation of the tension strap 30 is described with continuing reference to FIGS. 1(a & b), 3(a & b), 4(a & b) and 5(a & b). Prior to application of the tension strap 30, the knee brace 20 is first fastened to the wearer's leg 12 in a normal fashion. As shown in FIGS. 3(a & b), 4a and 5a, following the fastening of the knee brace 20 to the wearer's leg 12, the body 40 of the tension strap 30 is placed over the anterior portion of the knee 10 with the inner surface 42 of the body 40 and the guide pocket 62 in contact with the user's skin so that the buttress support member 60 lying adjacent to the lateral edge of the outline of the patella 14 such that the location orientation of the buttress support member 60 applies the desired directional support forces to the patella 14. The support straps (50L and 50M) are then inserted between the upper arms (22L and 22M) and lower arms (24L and 24M) of the knee brace 20 and the wearer's leg 12 and extended to the posterior side of the upper arms (22L and 22M) and lower arms (24L and 24M) of the knee brace 20. Self adhering patches of hook fastener material are disposed about the upper arms (22L and 22M) and lower arms (24L and 24M) of the knee brace 20 for vertically anchoring the support straps (50L and 50M) at the locations at which the distal ends (52L and 52M) of the support straps (50L and 50M) will be looped around the upper arms (22L and 22M) and lower arms (24L and 24M) of the knee brace 20.

As shown in FIGS. 1 (a & b), and 5 (a & b), the distal ends 52L of the support straps 50L oriented on the lateral side of the knee 10 and the patella 14 are then pulled around the outside of the laterally-oriented upper arm 22L and lower arm 24L of the knee brace 20 and looped from the posterior side of the laterally-oriented upper arm 22L and lower arm 24L of the knee brace 20 towards the anterior side of the upper arm 22L and lower arm 24L of the knee brace 20 such that the loop fastener material 56 disposed about the surface of the support straps 50L will adhere to the self adhering patches of hook fastener material 58 that have been disposed about the upper arm 22L and lower arm 24L of the knee brace 20. The distal ends 52L of the support straps 50L oriented on the lateral side of the knee 10 and the patella 14 are then interfastened to their respective support straps 50L or to the body 40 by adhering the patches of hook fastener material 54L affixed on the distal ends 52L of the support straps 50L to the loop fastener material 56 distributed about the proximal outer surface of each support strap 50L or the body 40. Upon doing so, the buttress support member 60 is anchored in the desired position about the lateral outline of the patella 14 so as to provide appropriate directional supporting forces to ensure proper tracking of the patella 14 during both extension and flexion of the knee 10.

As shown in FIGS. 1 (a & b), and 4 (a & b), the distal ends 52M of the support straps 50M oriented on the medial side of the knee 10 and patella 14 are then pulled in a medial direction until the desired tension across the support straps (50L and 50M) and the body 40 is achieved and such that the inner surface 42 of the body 40 and the guide pocket 62 remain in contact with the skin on the anterior side of the knee 10 and the patella 14. While maintaining this desired tension, the distal ends 52M of the support straps 50M oriented on the medial side of the knee 10 and the patella 14 are then pulled around the outside of the upper arm 22M and lower arm 24M of the knee brace 20 and looped from the posterior side of the upper arm 22M and lower arm 24M of the knee brace 20 towards the anterior side of the medially disposed upper arm 22M and lower arm 24M of the knee brace 20 such that the loop fastener material 56 disposed about the surface of the support straps 50M will adhere to the self adhering patches of loop fastener material 58 that have been disposed about the upper arm 22M and lower arm 24M of the knee brace 20. The distal ends 52M of the support straps 50M oriented on the medial side of the knee 10 and the patella 14 are then interfastened to their respective support straps 50M or the body 40, achieved in one embodiment by adhering the patches of hook fastener material 54M affixed on the distal ends 52M of the support straps 50M, depending on the amount of elastic deformation of the support straps 50M caused by the tension force applied to the support straps 50M, to the loop fastener material 56 distributed about the proximal exposed surface of each support strap 50M, the body 40, or, the exposed surface of the laterally disposed support straps 50L.

It is understood that the user may manipulate the level of directional support forces applied to the patella 14 by the tension strap 30 through the buttress support member 60 by increasing or decreasing the amount of tension applied when fastening the support straps (50L and 50M) in the manner described above. Thus, the amount of the directional forces applied to the patella 14 can be accurately adjusted for maximum efficiency and comfort of the wearer, and the tension strap 30 can be quickly applied without requiring any unusual skill.

Upon attaching the tension strap 30 in this manner, one or more appropriate directional forces are applied to the patella 14 by means of tension forces applied to the buttress support member 60. As shown in FIG. 10, one learned in the art will understand that a plurality of desirable directional forces can be supplied to the patella 14 by the buttress support member 60 by manipulating the locations at which the support straps (50L and 50M) are fastened about the upper arms (22L and 22M) and lower arms (24L and 24M) of the knee brace 20 in the manner described above.

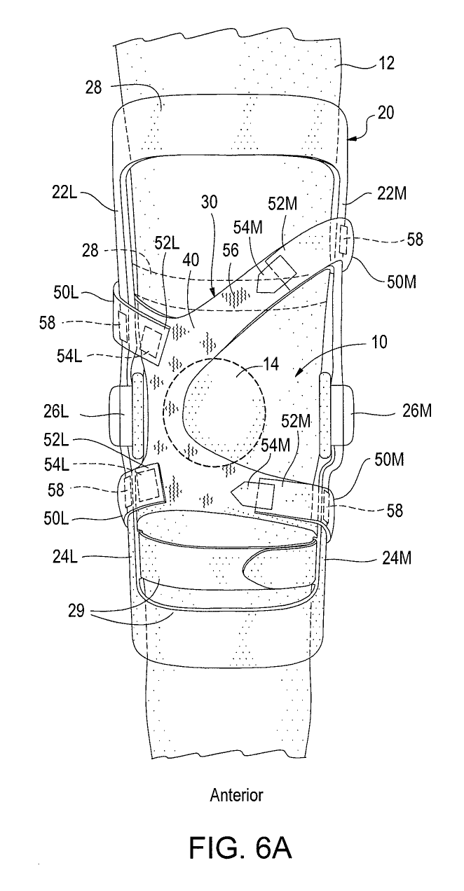

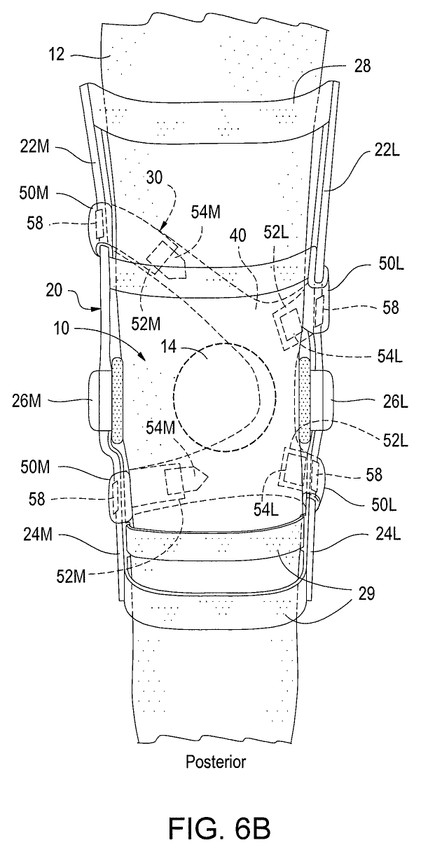

A second embodiment of the invention is shown in FIGS. 6 (a & b), 7 (a & b), 8 (a & b) and 9 (a & b) with the tension strap 30 having two primary parts: the body 40, which engages the skin on the anterior side of the wearer's knee 10 and patella 14; and a plurality of support straps (50L and 50M) (here shown as four in number) extending distally from the body 40 which engage the knee brace 20 at the distal ends (52L and 52M) of the support straps (50L and 50M) in the manner later described. As in the first embodiment of the invention, the body 40 is formed of a flexible, elastic material, such as, but not limited to neoprene with unbreakable looped backing (ULP) on the outer surface of the body 40. The inner surface 42 of the body 40, being that surface of the body 40 that will contact the skin on the anterior side of the wearer's knee 10 in the manner later described, is of a non-slip material, such as, but not limited to, exposed neoprene or synthetic shark skin.

As in the first embodiment, the support straps (50L and 50M) are formed of a flexible, elastic material, such as, but not limited to, neoprene with unbreakable looped backing (ULP) on both sides of the support straps (50L and 50M). In one embodiment, the support straps (50L and 50M) and body 40 are affixed together by sewing, however, one skilled in the art will understand that the support straps (50L and 50M) and body 40 may be of unitary construction or may be affixed together by other like means.

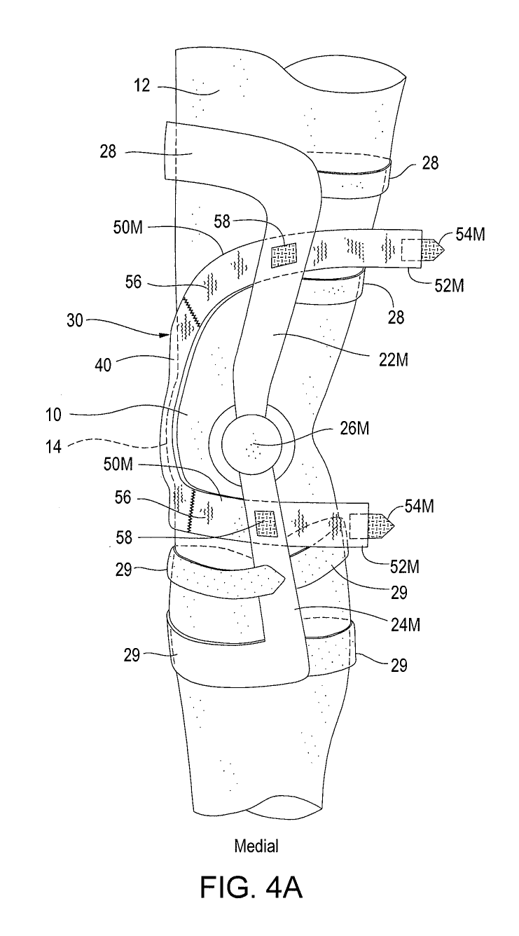

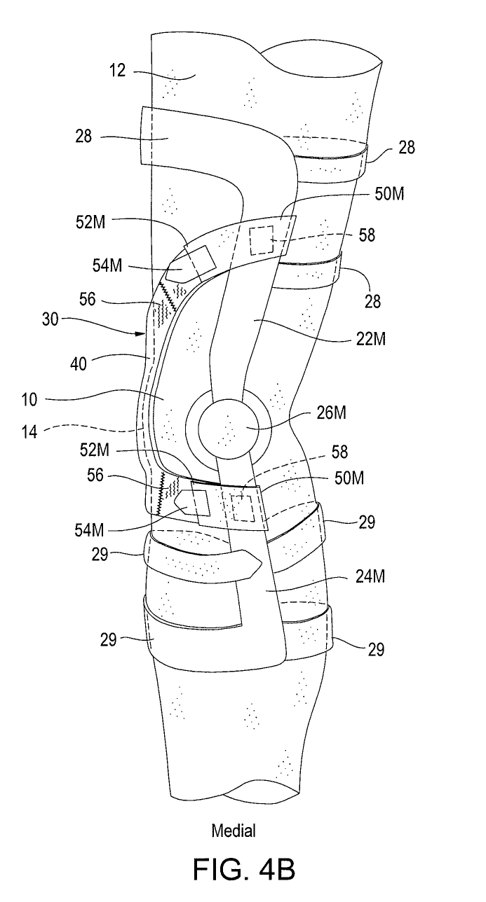

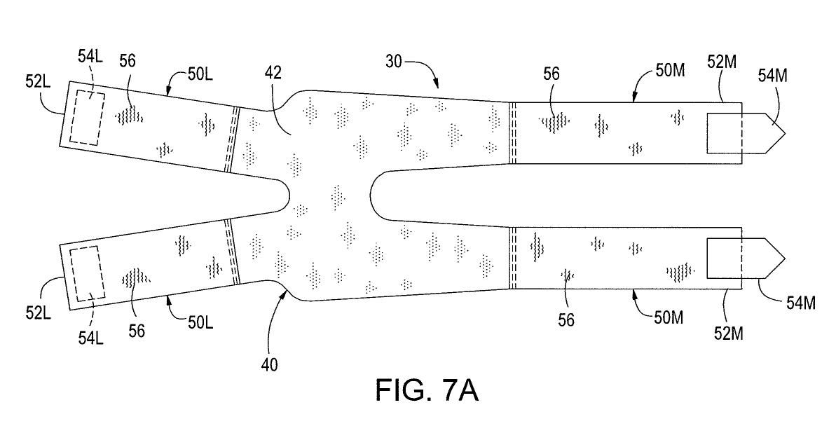

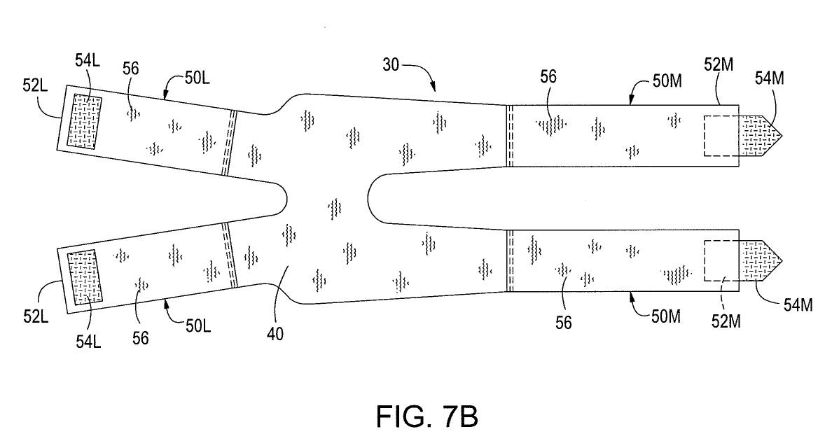

As shown in FIGS. 4 (a & b), 6 (a & b), 7 (a & b), 8 (a & b) and 9 (a & b), the lengths of the support straps (50L and 50M) are such to permit the distal ends (52L and 52M) of the support straps (50L and 50M) to be wrapped or looped about the medially and laterally disposed upper arms (22L and 22M) and lower arms (24L and 24M) of the knee brace 20 in the fashion later described. A patch of hook fastener material (54L and 54M) is affixed to the distal end (52L and 52M) of each support strap (50L and 50M). As shown in FIGS. 7 (a & b), in one embodiment, the outer dimensions of the patches of hook fastener material 54L affixed to the distal ends 52L of the support straps 50L that are laterally oriented are contained within the outer dimensions of the outer surface of the support straps 50L and may be affixed to the outer surface of the support straps 50L by such means as gluing, sewing or through the use of double sided hook fastener material. In one embodiment, the patches of hook fastener material 54M affixed to the distal ends 52M of the medially oriented support straps 50M append distally off the distal ends 52M of the support straps 50M and are affixed to the support straps 50M by use of alligator tabs also of hook fastener material that adhere to both the inner and outer surface of the support straps 50M. However, it will be appreciated that the patches of hook fastener material 50M may be affixed to the distal ends 52M of the support straps 50M by any manner of acceptable means.

As shown in FIGS. 6 (a & b), 8b and 9b, because loop fastener material 56 is distributed about the proximal surfaces of each support strap (50L and 50M) and the outer surface of the body 40, the support straps (50L and 50M) having been wrapped or looped about the medial and lateral upper arms (22L and 22M) and lower arms (24L and 24M) of the knee brace 20 in the fashion later described, upon closing the looped support straps (50L and 50M), the patch of hook fastener material (54L and 54M) of each support strap (50L and 50M) will adhere to the loop fastener material 56 of each support strap (50L and 50M) or the outer surface of the body 40, thereby securing the distal ends (50L and 50M) of the support straps (50L and 50M) about the upper arms (22L and 22M) and lower arms (24L and 24M) of the knee brace 20.

As shown in FIGS. 4 (a & b), 6 (a & b), 8 (a & b) and 9 (a & b), a self adhesive patch of hook fastener material (58) will also be applied to each of the upper arms (22L and 22M) and lower arms (24L and 24M) of the knee brace 20 in the desired positions along the upper arms (22L and 22M) and lower arms (24L and 24M) of the knee brace 20 where the support straps (50L and 50M) will be wrapped or looped about and secured to the upper arms (22L and 22M) and lower arms (24L and 24M) of the knee brace 20 so as to anchor the vertical positions of the support straps (50L and 50M) about the upper arms (22L and 22M) and lower arms (24L and 24M) of the knee brace 20.

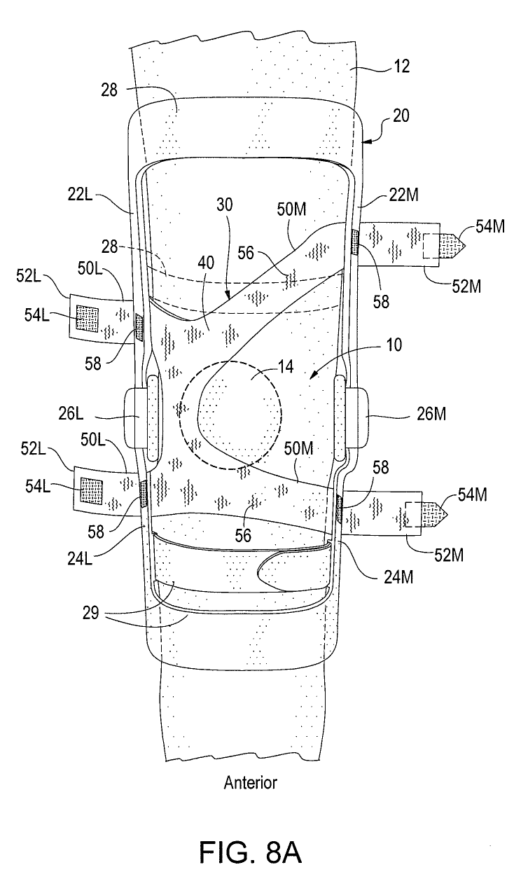

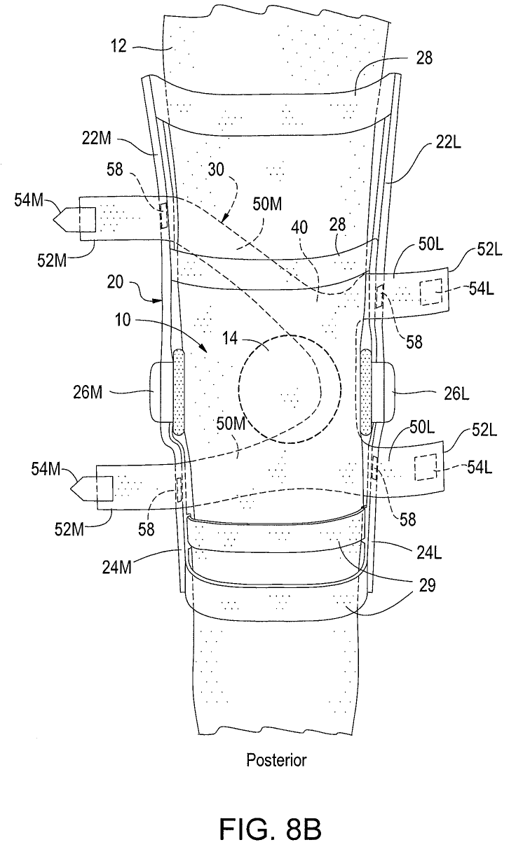

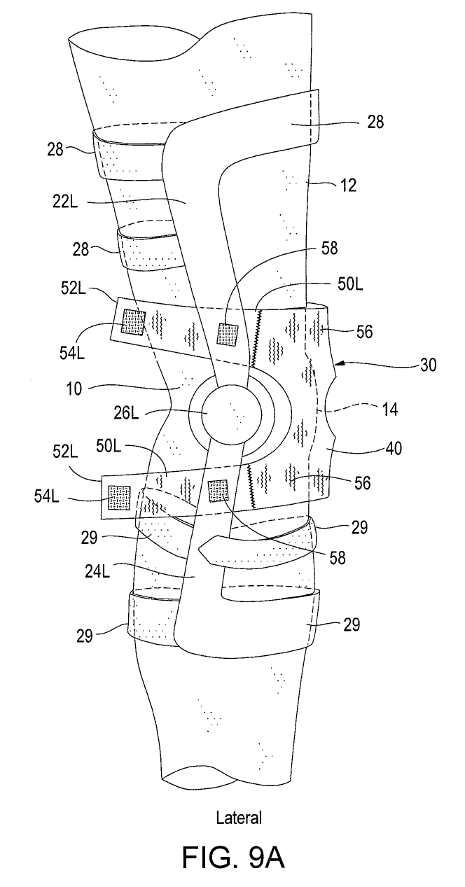

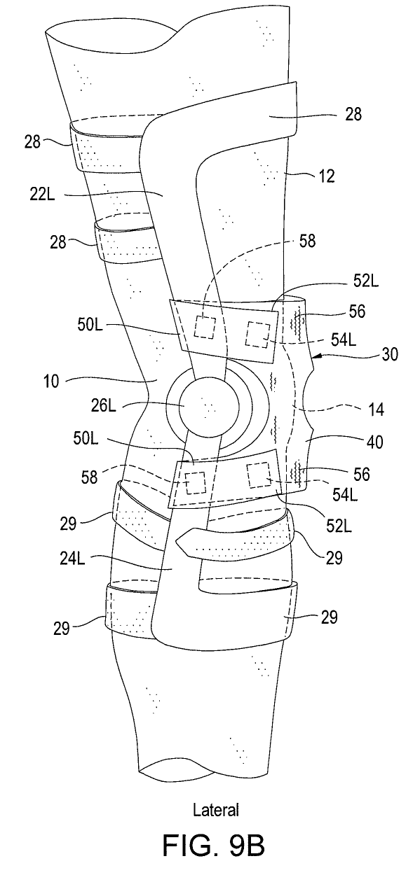

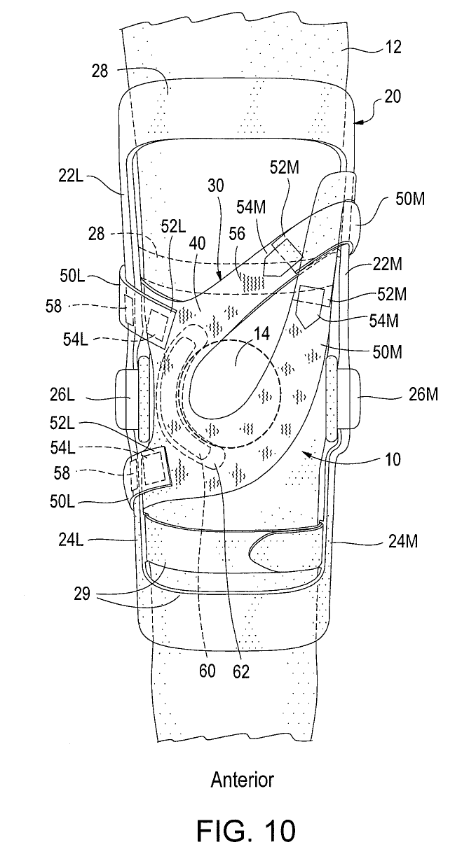

Operation of the tension strap 30 is described with continuing reference to FIGS. 4 (a & b), 6 (a & b), 8 (a & b) and 9 (a & b). Prior to application of the tension strap 30, the knee brace 20 is first fastened to the wearer's leg 12 in a normal fashion. As shown in FIGS. 4a, 8 (a & b) and 9a, following the fastening of the knee brace 20 to the wearer's leg 12, the body 40 of the tension strap 30 is placed over the anterior portion of the knee 10 with the inner surface 42 of the body 40 in contact with the skin on the anterior side of the wearer's knee 10 and patella 14. The support straps (50L and 50M) are then inserted between the upper arms (22L and 22M) and lower arms (24L and 24M) of the knee brace 20 and the wearer's leg 12 and extended to the posterior side of the upper arms (22L and 22M) and lower arms (24L and 24M) of the knee brace 20. Self adhering patches of hook fastener material 58 are disposed about the upper arms (22L and 22M) and lower arms (24L and 24M) of the knee brace 20 for vertically anchoring the support straps (50L and 50M) at the locations at which the distal ends (52L and 52M) of the support straps (50L and 50M) will be looped around the upper arms (22L and 22M) and lower arms (24L and 24M) of the knee brace 20.

As shown in FIGS. 6 (a & b) and 9 (a & b), the distal ends 52L of the support straps 50L oriented on the lateral side of the knee 10 and the patella 14 are then pulled around the outside of the laterally-oriented upper arm 22L and lower arm 24L of the knee brace 20 and looped from the posterior side of the laterally-oriented upper arm 22L and lower arm 24L of the knee brace 20 towards the anterior side of the laterally-oriented upper arm 22L and lower arm 24L of the knee brace 20 such that the loop fastener material 56 disposed about the surface of the support straps 50L will adhere to the self adhering patches of hook fastener material 58 that have been disposed about the upper arm 22L and lower arm 24L of the knee brace 20. The distal ends 52L of the laterally-oriented support straps 50L are then interfastened to their respective support straps 50L or the body 40 by adhering the patches of hook fastener material 54L affixed on the distal ends 52L of the support straps 50L to the loop fastener material 56 distributed about the proximal outer surface of each support strap 52L or the body 40.

As shown in FIGS. 4 (a & b) and 6 (a & b), the distal ends 52M of the support straps 50M oriented on the medial side of the knee 10 and patella 14 are then pulled in a medial direction until the desired tension across the support straps (50L and 50M) and the body 40 is achieved and such that the inner surface 42 of the body 40 is in contact with the skin on the anterior side of the knee 10 and the patella 14. While maintaining this desired tension, the distal ends 52M of the support straps 50M oriented on the medial side of the knee 10 and the patella 14 are then pulled around the outside of the medially-oriented upper arm 22M and lower arm 24M of the knee brace 20 and looped from the posterior side of the medially-oriented upper arm 22M and lower arm 24M of the knee brace 20 towards the anterior side of the medially-oriented upper arm 22M and lower arm 24M of the knee brace 20 such that the loop fastener material 56 disposed about the surface of the support straps 50M will adhere to the self adhering patches of hook fastener material 58 that have been disposed about the upper arm 22M and lower 24M of the knee brace 20. The distal ends 52M of the medially-oriented support straps 50M are then interfastened to their respective support straps 50M or the body 40, achieved in one embodiment by adhering the patches of hook fastener material 54M affixed on the distal ends 52M of the medially-oriented support straps 50M, depending upon the amount of elastic deformation of the support straps 50M caused by the tension force applied to the support straps 50M, to the loop fastener material 56 distributed about the proximal exposed surface of their respective support straps 50M, the body 40, or the exposed surface of the laterally disposed support straps 50L.

In both described embodiments of the present disclosure, through fastening the support straps (50L and 50M) of the tension strap 30 about the upper arms (22L and 22M) and lower arms (24L and 24M) of the knee brace 20 in the manner provided, each of the pair of polycentric hinge assemblies (26L and 26M) will be pulled inwardly towards each other about the joint of the knee 10 so as to provide resistance to vertical migration of the knee brace 20 about the wearer's knee 10. Furthermore, because the non-slip inner surface 42 of the body 40 (and the guide pocket 62 in the first embodiment) will be in continuous engagement with the skin of the wearer of the knee brace 20, further resistance to vertical migration of the knee brace 20 is provided during flexion and extension of the knee 10 while wearing the knee brace 20.

The construction of the tension strap 30 also reduces the need for large inventories in that only one size of tension strap 30 is required to fit the usual range of knee 10 and knee brace 20 dimensions. Further, while a knee brace 20 is typically constructed to fit either the right or the left leg 10, but not both, the construction of the tension strap 30 is universal such that it can be used with a knee brace 20 designed to fit either the left or right leg 10.

It is appreciated that various modifications to the inventive concepts may be apparent to those skilled in the art without departing from the spirit and scope of the invention.

What is claimed is:

1. A tension strap for use with a knee brace, said tension strap comprising:
a body having an inner surface and an outer surface;
a plurality of support straps, each of said support straps having a proximal end in communication with said body and a free distal end; and
means for adjustably securing said tension strap to the knee brace.

2. The tension strap of claim 1 wherein said body and said support straps are of a stretchable, elastic material.

3. The tension strap of claim 2 where the material is neoprene.

4. The tension strap of claim 1 wherein said support straps are formed integral with said body.

5. The tension strap of claim 1 wherein said inner surface is of or lined with a non-slip material and wherein at least a portion of said inner surface will be in direct engagement with a portion of the skin on the anterior side of the knee and patella so as to provide resistance to vertical migration of the knee brace about the area of the knee.

6. The tension strap of claim 1 wherein said means for adjustably securing comprises removably securing said support straps to the knee brace by looping said distal ends of said support straps around the knee brace and a means of interengagement of each said distal end of said support straps with its respective support strap or said outer surface of said body.

7. The tension strap of claim 6 wherein said interengagement means comprises hook fastener material positioned on said distal ends of said support straps and loop fastener material positioned on said support straps and on said outer surface of said body or loop fastener material positioned on said distal ends of said support straps and hook fastener material positioned on said support straps and on said outer surface of said body.

8. The tension strap of claim 6 wherein said means for adjustably securing further comprises a loop fastener material disbursed about said tension straps and a hook fastener material removably positioned on the knee brace, such that when said distal ends of said support straps are looped about the knee brace, said hook fastener material will engage the loop fastener material so as to control movement of said support straps about the knee brace.

9. The tension strap of claim 1 further comprising a support buttress member affixed to said body, wherein said support buttress member is constructed to contact at least a portion of the circumference of the patella, the support buttress member providing a supporting directional force to the patella, maintaining proper tracking of the patella during extension and flexion of the knee joint or a combination of the foregoing.

10. The tension strap of claim 9 wherein the locations at which said support straps are secured to the knee brace can be manipulated such that said support buttress member can be positioned in a plurality of locations about the patella such that a plurality of said supporting directional forces can be applied to the patella by said support buttress member.

11. The tension strap of claim 9 where the support buttress member is contained within a guide pocket secured to the body.

12. A tension strap for use with a knee brace, said tension strap comprising:
a. a body having an inner surface and an outer surface, the outer surface comprising a first reversible engaging element on at least a portion of its surface;
b. a plurality of support straps, each of said support straps having an inner surface, an outer surface, a proximal end in communication with said body and a free distal end, the support straps comprising a second reversibly engaging element complementary to the first reversible engaging element along at least a portion of the inner surface;
c. wherein the tension strap are reversibly secured to said knee brace by looping said distal ends of said support straps around the knee brace such that the first and second reversible engaging elements engage one another to reversibly secure the tension strap to the knee brace to prevent vertical migration of the knee brace about the area of the knee.

13. The tension strap of claim 12 where the second reversible engaging element is positioned on the distal ends of said support straps.

14. The tension strap of claim 12 further comprising a support buttress member affixed to said body, wherein said support buttress member is constructed to contact at least a portion of the circumference of the patella, the support buttress member (i) providing a supporting directional force to the patella, (ii) maintaining proper tracking of the patella during extension and flexion of the knee joint, (iii) or a combination of the foregoing.

15. The tension strap of claim 12 where the first and second reversible engaging elements are hook and loop fastener material.

16. The tension strap of claim 12 further comprising a third reversible engaging element removably positioned on said knee brace, the third reversible securing element being complementary to the first reversible engaging element, the second reversible engaging element or both, the third reversible engaging element engaging at least one of the first or second reversible engaging elements to control the movement of the support strap about the knee brace.

17. The tension strap of claim 16 where the first, second and third reversible engaging elements are hook and loop fastener material.
